(19) United States
(12) Patent Application Publication
(10) Pub. No.: US 2024/0000340 A1
(43) Pub. Date: Jan. 4, 2024

(54) NONBINARY RESPIRATORY INDICATION OF PHYSIOLOGICAL CONDITIONS

(71) Applicant: Calibre Biometrics Inc., Wellesley Hills, MA (US)

(72) Inventor: Udi E. Meirav, Waban, MA (US)

(73) Assignee: Calibre Biometrics Inc., Wellesley Hill, MA (US)

(21) Appl. No.: 18/469,929

(22) Filed: Sep. 19, 2023

Related U.S. Application Data
(63) Continuation of application No. PCT/US22/21530, filed on Mar. 23, 2022.
(60) Provisional application No. 63/165,839, filed on Mar. 25, 2021.

(51) Int. Cl. A61B 5/08; A61B 5/091; A61B 5/087; A61B 5/11

(52) U.S. Cl. CPC A61B 5/082; A61B 5/091; A61B 5/087; A61B 5/11

(57) ABSTRACT

Embodiments of the invention are generally directed to using breath measurement to identify certain physiological states, conditions and disorders. In one example, breath measurement may be used in producing a non-binary indicator of the likelihood or extent to which a subject is experiencing or approaching ketosis. Other metabolic or respiratory states may be indicated and/or identified.

### RELATED APPLICATIONS

This application is a continuation of commonly assigned International (PCT) Application No. PCT/US2022/021530, filed Mar. 23, 2022, entitled “Nonbinary Respiratory Indication of Physiological Conditions,” bearing Attorney Docket No. V0340.70003W000, which claims the benefit of the filing date of commonly assigned U.S. Provisional Patent Application Ser. No. 63/165,839, filed Mar. 25, 2021, entitled “Method And System Of Tracking And Indicating Ketosis States,” bearing Attorney Docket No. V0340.70003US00. Each of the documents listed above is incorporated herein by reference in its entirety.

### TECHNICAL FIELD

Embodiments of the present disclosure generally relate to systems, methods and devices for collecting, analyzing and utilizing respiratory, physiological, metabolic or biometric data.

### BACKGROUND

Monitoring the volume and composition of exhaled breath can be useful for various diagnostic and biometric applications including medical, sports and nutrition. While the practicalities of breath measurement have limited the broader adoption of such techniques, their physiological validity and accuracy is well established and accepted by medical professionals and scientists. Among the parameters that can be determined from such tests, the amount of carbon dioxide production and oxygen consumption, as well as their ratio (the respiratory exchange ratio or RER) are routinely measured and relied upon to quantify the metabolic energy production rate and the mix of metabolic fuels used to produce this energy.

Advances in low cost, portable breath-measurement devices are bringing new opportunities to the extraction of useful information from breath, including metabolic indicators and medical diagnostics. These system measure real time respiratory oxygen consumption and CO2 production as well as overall air flow. Readings are transmitted to portable devices that can calculate and display various respiratory-derived quantities that are useful or interesting to individual users as well as trainers and medical professionals, such as real time energy production (i.e. calories per minute, etc.) or minute volume rate (i.e. liters of air per minute).

Ketosis describes is a metabolic state where ketones such as acetoacetate, beta-hydroxybutyrate and acetone—which the body derives from metabolizing stored fats—circulate in the blood and serve as the primary energy fuel, instead of glucose, for muscles and other organs in the body. Nutritional ketosis occurs naturally in healthy individuals when the body exhausts its limited stores of carbohydrates and glycogen. There is growing interest in the benefits of deliberately inducing and maintaining a state of ketosis, through extended fasting or through so-called ketogenic diets that strictly reduce carbohydrate ingestion.

More broadly, there a various physiological states, conditions and disorders of the human body that are typically described in binary terms—the condition is either present or not—even though their onset is not truly binary but gradual.

### BRIEF DESCRIPTION OF DRAWINGS

FIG. 1 is a chart depicting a representative respiratory ketosis “score” in relation to a respiratory exchange ratio, in accordance with some embodiments of the invention;

FIG. 2 is a flow chart of a representative process for generating such a score from two independent quantities derived from breath measurement, in accordance with some embodiments of the invention; and

FIG. 3 is a block diagram depicting a representative computing system which may be used in producing a non-binary indicator of a physiological or metabolic condition, state or disorder, in accordance with some embodiments of the invention.

### DETAILED DESCRIPTION

The Assignee has appreciated that in some cases, breath measurement may offer unique and independent ways of identifying some physiological states, conditions and disorders, and that as breath measurement becomes easier and more widespread, it may offer new insights into such states and conditions.

As one example, one of the challenges faced by individuals trying to maintain ketogenic regimens is how to know when their body is in fact in this metabolic state. The tools available to individuals to do so are inconvenient and inaccurate, most commonly using detection of ketone bodies in the blood, which requires drawing a blood sample every time a reading is sought.

Nutritional ketosis is understood not just as a metabolic process but as a metabolic state where the body shifts its energy reliance to ketosis and fat metabolism, often discussed in terms of a crossover or a binary condition. Aside from the question of how sharp the threshold or onset is, there is a growing need for new tools to help monitor this condition and the transition into it.

Ketosis is but one example of an array of physiological or metabolic conditions, some of which are part of normal healthy physiology, others perhaps not, where many individuals stand to benefit from having new and better tools to monitor their condition. Notwithstanding the conditions being described as present or absent, the onset may be gradual and partial.

In certain embodiments an indication of whether the person (interchangeably referred to as the subject or individual), is in a state of ketosis can be derived from breath measurement and analysis. There are several respiratory parameters that can measured, including the rate of air flow, the breath volume, breath rate (frequency) or duration—collectively respiratory parameters, as well as concentration of oxygen (O2) and carbon dioxide (CO2). Respiratory parameters can be determined using a flow sensor or a differential pressure sensor along the path of respiratory air flow, and a breath collection device, for example a breathing mask worn by the person, that direct the person's respiratory air flow. In certain embodiments the mask is configured to cover the mouth and the nostrils and direct air flow through designated inlets or outlets (which can be tubes or apertures) so that all breath flows through these inlets and outlets

Measuring the concentration of 02 and CO2 requires gas sensors specific to these particular gases, configured to measure the exhaled air. Although this can be done by locating the sensors along the flow path of exhaled air, it may be required in some embodiments to separate the exhaled air flow that from that of inhaled air to ensure that the concentration measured is specific to exhaled air. The rate of oxygen consumption, VO2 (also known as “oxygen minute volume” and expressed in standard liters per minute) is the net difference between the amount of oxygen inhaled and the amount exhaled per minute, which in turn are the product of a respiratory volume and the oxygen concentration corresponding to inhaled and exhaled breath. Similarly the CO2 minute volume, VCO2, is the net difference between the amount exhaled and the amount inhaled in the same time period, although the amount of inhaled CO2 is usually negligible. The ratio between VCO2 and VO2 is called the respiratory exchange ratio (RER), which can be calculated once VO2 and VCO2 are known.

Ketosis is generally associated with an RER value that is very close to a limit value (denoted as KLV) of approximately KLV≈0.7, which is the RER characteristic of fatty acid catabolism. By contrast, carbohydrate catabolism has RER=1, so even a partial amount of carbohydrate utilization is under way, the RER is greater than KLV and ketosis is less likely to be the dominant contribution to energy metabolism. Thus RER measurement could reasonably indicate a likelihood of whether the body is in ketosis.

In this framework, a binary “ketosis indicator” (labeled as KI) can be derived from the value of RER as follows:
If {RER is greater than KLV} then {KI=no}; else {KI=yes}

This represents a binary view of ketosis and in some embodiments it can serve as a coarse indicator for likelihood of ketosis. However, this binary logic will not always yield a good real-time ketosis monitoring tool in practice, for a number of reasons. Since RER is derived as a ratio between the VO2 and VCO2, the accuracy of RER measurement is limited by the measurement error of these quantities. These are cumulative errors due to a variety of factors including but not limited to the finite calibration accuracy, repeatability of gas concentration sensors (both oxygen and CO2) and additional uncertainties, for example from the need to subtract the exhaled oxygen from the inhaled oxygen to determine the actual net consumption. This netting out involves factors like the different temperature and humidity levels of inhaled air and exhaled air, for example, further affecting the absolute accuracy of VO2 and VCO2, and therefore RER. Even temporal electronic noise would create a misleading indication of bouncing in and out of ketosis that is not based in metabolic reality but only in the limitations of the measurement system.

While measurement errors are ubiquitous in all walks of life, the ramifications for a binary KI are significant and are not solvable simply by an adjustment to the threshold KLV value. If the theoretically correct threshold value (assumed to be 0.7) is used and a reading has a small inaccuracy of a few percent in the positive direction, the result may be that it will never produce a “yes” value for KI, regardless of the true underlying metabolic state. If the threshold is moved to a higher value, e.g. 0.75, the result could be that a significant fraction of the “yes” readings are incorrect, and a departure from ketosis is not flagged promptly. In other words, any small error in RER can generate a 100% error in KI.

Beyond the physical measurement accuracy, the relationship between ketosis and RER is strong but not binary, where other factors can affect RER and KLV, at least temporarily. While the scientific understanding of these factors continues to evolve, at a binary reading based on a cutoff (whether at RER=0.7 or a slightly different value) leads to a potentially false result in a significant fraction of cases.

In one embodiment, a solution is proposed as a method of data analysis and presentation that introduces a non-binary “score” for ketosis, which in the following will be called a respiratory ketosis score (RKS).

In one embodiment the RKS can take any value between 0 and 1 (or 100%). The value of RKS approaches zero as the value of RER gets further away from the benchmark value of KLV; and approaches 1 (or 100%) as RER gets closer to the benchmark. There can be several possible meanings or interpretations of the score.

The score can be interpreted in a number of possible meanings that are related but different. In some embodiments ketosis is still viewed in binary terms but the score is understood as an estimated probability of the person being in (or out of) ketosis. In this type of embodiment a score of 0.3 is understood as a 30% probability that the person is in a state of ketosis and 70% that they are not. In some embodiments the score more loosely suggests a “likelihood” but without necessarily prescribing a valid, mathematically rigorous probability. In some embodiments the score is interpreted not in terms or a probability of a binary state, but rather as a measure of the relative degree of significance of ketosis as part of the overall energy metabolism. In some embodiments the score serves as a gauge or indicator tracking gradual transitions between two states, namely between a state where ketosis is relatively insignificant to a state where ketosis is significant or even dominant.

Regardless of its preferred suggestive interpretation, one of the benefits of this non-binary score is that a small measurement error (in RER or in other quantities) will generally produce a commensurately small error in the score, as opposed to a complete true/false error in a binary-valued parameter like KI.

In this embodiment the boundary values of measured RER associated with RKS=0 and RKS=1 can be adjusted to address measurement errors or user preferences. A non-limiting example would be 0.7 of RKS=1 (ketosis very likely) and 0.75 for RKS=0 (ketosis unlikely), where intermediate values are calculated by linear interpolation.

In some embodiments, the score is a finite set of discrete values including but not limited to integers or percentages. In some embodiments the score is a descriptor or a label, such as words (including but not limited to “POSITIVE”, “LIKELY”, “POSSIBLE”, “UNLIKELY”, “NEGATIVE”, “HIGH”, “LOW” and so forth), names, or any alphanumeric combination. In some embodiments the score comprises a range or set of colors, shapes, or a set of icons or graphical representations that can be displayed, such as on a screen. It is to be understood that these values are merely a non-limiting example and that a non-binary indication may be implemented or represented in any of numerous ways.

The measured values of RER are mapped to RKS values using any ordinary method including but not limited to a logical rule, a calculation or a lookup table. In one embodiment the range of RER values is mapped to RKS values through a piecewise linear calculation:

$$RKS = \begin{matrix}1 & for RER \leq 0.7 \\ 10 \times ( 0.8 - RER ) & for 0.7 \leq RER \leq 0.8 \\ 0 & for RER \geq 0.8\end{matrix}$$

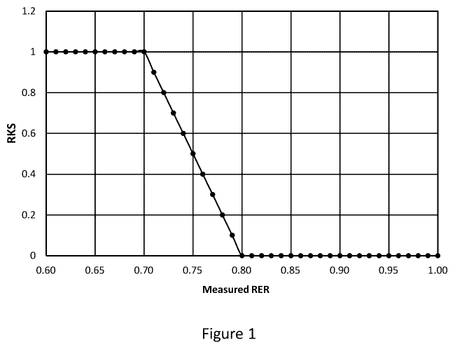

This can be interpreted to suggest the likelihood of ketosis as gradually increasing as RER decreases, from a value of zero (ketosis is unlikely) when RER is greater than 0.8 to a value of 1 when it is 0.7 or lower (ketosis is likely). FIG. 1 is a chart illustrating this mathematical relationship between RER and RKS associated with this embodiment. This example is merely for illustration and not intended to suggest that the range values are right, nor that the score needs to be piecewise linear.

In certain embodiments, a table is user to convert RER to a discrete set of score values. In one non-limiting example, each entry or row in the table corresponds to a range of values of RER associated with that score, represented by a minimum and maximum value of RER for that particular range, e.g. from 0.72 to 0.73. In this example each row specifies a RKS (score) value—numeric or other—that is assigned to any RER reading that falls within that specified range. An illustrative example of such a table is shown in Table 1 and Table 2. The ranges can be spelled out explicitly with a Min and Max value, but in some embodiments the Max value can be implied by the Min of the next range.

TABLE 1

RER Range

| Min | Max | Score |
|---|---|---|
| 0 | 0.68 | 10 |
| 0.68 | 0.70 | 9 |
| 0.70 | 0.71 | 8 |
| 0.71 | 0.72 | 7 |
| 0.72 | 0.73 | 6 |
| 0.73 | 0.74 | 5 |
| 0.74 | 0.75 | 4 |
| 0.75 | 0.76 | 3 |
| 0.76 | 0.77 | 2 |
| 0.77 | 0.78 | 1 |
| 0.78 | (no upper limit) | 0 |

TABLE 2

RER Range

| Min | Max | Score |
|---|---|---|
| 0 | 0.7 | POSITIVE |
| 0.7 | 0.75 | LIKELY |
| 0.75 | 0.8 | UNLIKELY |
| 0.8 | No upper limit | NEGATIVE |

In some embodiments the RKS is further determined or influenced by one or more additional variables that are not derived exclusively from the current value of RER. In some embodiments these influences can be related to values of RER measured at other times or to values from other sensors.

In some embodiments values derived from other sensors can be used in conjunction with, or even or instead of, RER to determine a more reliable score. This can help generate a more reliable “score” for any number of reasons. A non limiting example is physical exertion, which can temporarily elevate RER, even while the person remains in a state of nutritional ketosis. In some embodiments the sudden increase in RER due to exercise is intentionally not associated with a reduction in the likelihood of ketosis. This can be done by detecting physical exertion through one or more additional variable, including but not limited to heart rate, breath rate, breath volume, and motion sensing, and incorporating that additional variable in the determination of RKS.

In a non-limiting example, other respiratory sensors can detect breath rate or frequency (expressed in breaths per minute or bpm), instantaneous breath flow rate (liters per second), exhaled minute volume (liters per minute) and breath volume (liters per breath)—examples of what will be collectively termed respiratory parameters—which are influenced by exertion. Intense exertion is often associated with an increase in the values of certain respiratory parameters and this information can be utilized for a more accurate indication of whether or not the RER should still be correlated with ketosis. In some embodiments this is addressed by changing the functional dependence of the score on the one or more respiratory parameters. In one embodiment such functional change can be change in the slope or intercepts of the linear section of the RKS dependence on RER. In some embodiments it is a change in the values or ranges of RER corresponding to a particular RKS value.

In one embodiment, a breath measurement apparatus comprises a mask worn by a subject and a number of sensors configured to measure exhaled O2 and CO2 concentrations and a pressure or flow sensor configured to detect breath flow, such that the apparatus can correctly determine the breath rate and the RER. The combination is RER and breath rate are then used to determine a respiratory ketosis score (RKS).

The following is an example of a numerical RKS, expressed algebraically in terms of RER and breath rate (BR) as follows:

RKS=Minimum{1,Maximum[0,1−100×(RER−0.7)/BR]}

In this example, for RER=0.7, RKS=1 (or 100%) regardless of the value of BR. However if RER=0.8 and BR=10 bpm, then RKS=0; but for the same value of RER=0.8, if the breath rate is doubled to BR=20, then RKS=0.5. This example is not intended as a prescriptive or optimal but only serves to illustrate how an additional variable like BR can be incorporated to modify RKS.

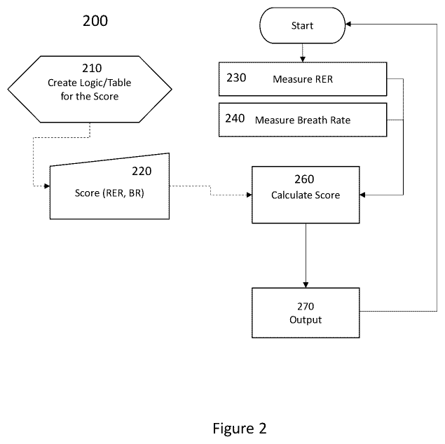

FIG. 2 depicts a flow chart representing an embodiment similar to the one just described. A fixed table or formula is first created (210) and stored (220); this table converts combinations of RER and BR values to a score. One or more digital breath measurements of RER (230) and BR (240) are performed on a person, and each time values of RER and BR are received, they are used along with the stored table or formula (220) to produce a score (260) which is then exported (270) as output to a display or to storage.

For non-numerical scores (e.g. LOW/MEDIUM/HIGH) it is also possible to incorporate an additional variable such as breath frequency. As a merely illustrative example, if RKS has a value of “MEDIUM” at RER=0.75 and a breathing rate of under 20 breaths per minute, that same value (“MEDIUM”) is also attained at RER=0.8 and 30 breaths per minute.

In some embodiments, a plurality of recently-measured values are factored in when breathing rate changes. For example, if the RKS has a value of “HIGH” with slow breathing, and subsequently some respiratory parameters increase significantly (indicating onset of stress or exercise), then a concomitant increase of RER is ignored insofar as making any changes to RKS. In other words, for this embodiment, in the case of a user already in ketosis before exercise, an increase in RER coinciding with an increase of respiration is not interpreted as a reset of the ketosis state and therefore does not update the value of RKS.

In some embodiments, upon a rapid increase in respiratory parameters the value of RKS is not re-calculated but rather labeled as “unknown” or “not applicable” or similarly neutral label. In some embodiments, when this happens, there is no displayed value at all, implying that during elevated respiration one cannot reliably use breath to impute the likelihood of nutritional ketosis.

In a separate example, sensors that detect traces of volatile organic compounds (VOCs) or any other bio-effluents in the breath are included in the determination of RKS. Trace VOCs such as such as ketones and aldehydes are known to be associated with ketosis, but their low concentrations and variability means they are not always detectable with reliability and accuracy. However, in a probability-based score such as RKS, an appropriate weight can be given to the detection of these VOCs if they are being tracked and sensed, for example increasing the RKS when one or more of the relevant VOCs are detected, commensurately with the amount detected.

In some embodiments, non-respiratory physiological and mechanical sensor readings and biometric variables can be collected concurrently and incorporated into the determination of the likelihood of ketosis. Such sensors and variables may include, but are not limited to, heart rate, electro-cardio signals, blood pressure, blood oxygen saturation, blood glucose, blood ketones, electrical conductivity, motion sensors/accelerometers, and electro-chemical sensors. In one embodiment the heart rate is sensed by a sensing device worn on the wrist or finger of the subject (in addition to a breath measurement device) and the measured heart rate is used to modulate the score along with the RER, in a way that is analogous, though not necessarily mathematically identical, to the that of a breath rate.

In some embodiments the determination of a value of a score like RKS from the RER may can rely on a plurality of recent RER measurements. In some embodiments using a plurality of measurements can lead to more reliable determination of ketosis, by removing noise, taking into account averages, rates of change or other trends, recognizing temporal variation patterns, or any other mathematical refinements. In a non-limiting example, average RER over an extended period of time is used instead of the instantaneous value, and outlier values may be filtered out before the averaging. In this example a short-lived increase, or decrease, in RER can be entirely ignored.

In some embodiments the rate of change of RER is used, in terms of an amount of change per unit time, such as change per minute or per second. For example, the RKS is higher—stronger indication of ketosis—when RER is close to KLV and stable over time, namely not changing very much over the last minute or 10 minutes. More generally this is a differential dependence of RKS on RER. In some embodiments, the average value of RER over a certain period is used in the calculation of RKS. This is an integral dependence. In a nonlimiting example, these types of analytic dependencies can help remove certain types of errors due to measurement glitches or rapid changes in the user's breathing that generate spurious temporary changes in RER.

### Beyond Ketosis

The method of obtaining and presenting a breath-derived non-binary score for a condition that is ordinarily represented or diagnosed in binary terms can be generalized and extended to other physiological, respiratory or metabolic conditions that are indicated in breath.

A non-limiting example of such a condition is anaerobic metabolism. When a physical effort reaches intensity that requires more energy than the body is able to generate through ordinary oxidative (aerobic) metabolism—typically when the muscles' oxygen requirement exceeds the capacity of the cardio-respiratory system—muscles turn to anaerobic metabolism, which is characterized, among other things, by generation of lactate and an RER that is greater than 1. This has the effect of an oxygen deficit and the buildup of lactate. Identifying in real time when an individual is crosses over into an anaerobic effort is not always easy but can be very useful information for athletic training.

Similarly to the case of ketosis, the presence of measurement uncertainties and physiological variability in the context of a binary distinction between two states creates a potential of an amplified error and reduced usefulness. Thus, a non-binary respiratory “anaerobic score” (RAS) can be calculated, recorded and displayed. In this example the RAS is the anaerobic equivalent of the RKS for ketosis. Its mathematical dependency on RER can, without limitation, be analytical or derived from a lookup table, and its value can be numerical or associated with a discrete list that includes, but is not limited to, words, numeric values, alpha-numeric strings, colors, and graphical representations.

In further analogy to the case of RKS, the determination of RAS can further rely on other measured quantities including, but not limited to, a respiratory rate, a respiratory volume, a breath volume, a heart rate, a systolic blood pressure level, a blood oxygen saturation level, and any other tracked biometric quantities or sensor readings. In some embodiments, the determination of RAS can further rely on differential and integral properties of RER or of other separately measured variables and readings. In contrast to RKS, however, exercise is in fact positively correlated with the likelihood of anaerobic metabolism so the RAS may be increased, rather than suppressed or decreased, when exertion is detected through one or more secondary variables like breath rate or heart rate.

In some embodiments, other metabolic or respiratory states can be identified with a non-binary score or metric. A non-limiting example of such states is post-anaerobic recovery, which occurs normally after buildup of lactate and in which proportionately high oxygen intake—namely low RER—may occur as the body restores its metabolic balance and eliminates metabolic products such as lactate. In the case of recovery, the determination of a “score” or a likelihood can be influenced not only by the value or RER and other biometric quantities but also on the recency of an effort level that is likely to have required temporary anaerobic metabolism.

The general method can also be applied to other medical conditions and disorders that have potential indication in breath. Such conditions may include, but are not limited to, Diabetes, Pre-diabetes, and Metabolic Syndrome. Alternatively it can be applied to the indication of neurological, psychological and mental states, including the various forms and stages of sleep.

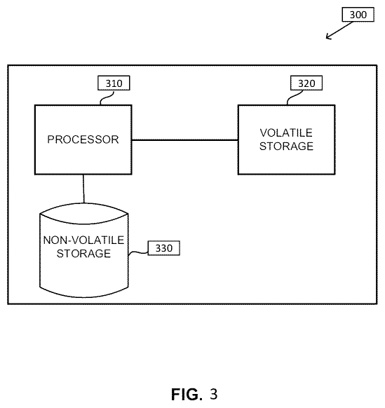

It should be appreciated from the foregoing that some embodiments of the invention may include a computing system configured to perform the techniques disclosed herein for determining a non-binary indication of a physiological state, condition or disorder (e.g., ketosis). A representative computing system 300 is shown in FIG. 3. One or more computing systems such as computer system 300 may be used to implement any or all of the functionality described above. The computing system 300 may include one or more processors 310 and one or more tangible, non-transitory computer-readable storage media (e.g., volatile storage 320 and one or more non-volatile storage media 330, which may be formed of any suitable non-volatile data storage media). The processor 310 may control writing data to and reading data from the volatile storage 320 and the non-volatile storage device 330 in any suitable manner, as the aspects of the present disclosure are not limited in this respect. To perform any of the functionality described herein, the processor 310 may execute one or more instructions stored in one or more computer-readable storage media (e.g., volatile storage 320), which may serve as tangible, non-transitory computer-readable storage media storing instructions for execution by the processor 310.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computing system or distributed among multiple computing systems. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of embodiments of the present disclosure comprises at least one computer-readable storage medium (i.e., a tangible, non-transitory computer-readable medium, such as a computer memory, a floppy disk, a compact disk, a magnetic tape, or other tangible, non-transitory computer-readable medium) encoded with a computer program (i.e., a plurality of instructions), which, when executed on one or more processors, performs above-discussed functions of embodiments of the present disclosure. The computer-readable storage medium can be transportable such that the program stored thereon can be loaded onto any computer resource to implement aspects of the present disclosure discussed herein. In addition, it should be appreciated that the reference to a computer program which, when executed, performs any of the above-discussed functions, is not limited to an application program running on a host computer. Rather, the term “computer program” is used herein in a generic sense to reference any type of computer code (e.g., software or microcode) that can be employed to program one or more processors to implement above-discussed aspects of the present disclosure.

The phraseology and terminology used herein is for the purpose of description and should not be regarded as limiting. The use of “including,” “comprising,” “having,” “containing”, “involving”, and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements from each other.

Having described several embodiments of the disclosure in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. A method of determining a likelihood that a subject is in, or approaching, ketosis, the method comprising acts of:
(a) measuring exhaled breath and determining a respiratory exchange ratio (RER) of the subject;
(b) using a sensing device to determine whether the subject is in a state of exertion or has a physiological condition which affects the correlation between RER and the likelihood that the subject is in ketosis; and
(c) taking at least one measurement of the sensing device from act (b), and using the RER measured in the act (a) to determine a score that is associated with the likelihood that the subject is in ketosis, wherein the score is one of a predetermined set of possible values.

2. The method of claim 1 where the set of possible scores comprises numerical values within a range between a minimum value and a maximum value.

3. The method of claim 1 where the set of possible scores comprises one or more of a percentage, a word, a name, an alphanumeric label, a graphic representation, an icon, and a color.

4. The method of claim 1 where the sensing device measures of flow, a rate, a frequency, or a volume of breath.

5. The method of claim 1 where the sensing device detects a concentration of a bio-effluent in the exhaled breath associated with ketosis, including but not limited to ketones.

6. The method of claim 1 where the sensing device is in contact with the subject's skin.

7. The method of claim 1 where the sensing device measures motion or acceleration.

8. The method of claim 1 where a plurality or sequence of values of the RER or of the sensing device, measured over a certain time interval, are used in determining a score.

9. A method of determining a likelihood that a subject is in, or approaching, a physiological state, condition or disorder, method comprising:
(a) measuring exhaled breath and determining a respiratory exchange ratio (RER) of the subject;
(b) using a sensing device to measure an indicator of the subject's degree of exertion, physical activity, or excitation; and
(c) taking at least one measurement of the sensing device from act (b), and using the RER measured in the act (a) to determine a score that is associated with the likelihood that the subject is in the physiological state, wherein the score is one of a predetermined set of possible values.

10. The method of claim 9 where the condition is one of an anaerobic metabolism or post-anaerobic recovery.

11. The method of claim 9 where the condition is a metabolic irregularity including but not limited to ketosis, diabetes, pre-diabetes and metabolic syndrome.

12. The method of claim 9 where the condition is neurological or sleep related.

13. The method of claim 9 where the sensing device measures a flow, a rate, a frequency or a volume of breath or a presence of a particular bio-effluent in the breath.

14. The method of claim 9 where the sensing device is in contact with the subject's skin.

15. The method of claim 9 where the sensing device is mechanical, electrical or optical.
